(19) United States
(12) Patent Application Publication
(10) Pub. No.: US 2025/0101056 A1
(43) Pub. Date: Mar. 27, 2025

(54) SWEETENER COMPOUNDS, sweetened compositionS, and methods of sweetening food products, beverages and the like

(71) Applicant: Miralogx LLC, Tampa, FL (US)

(72) Inventor: Jonnie R. Williams, SR., Sarasota, FL (US)

(21) Appl. No.: 18/891,681

(22) Filed: Sep. 20, 2024

Related U.S. Application Data
(60) Provisional application No. 63/540,778, filed on Sep. 27, 2023.

(51) Int. Cl. C07H 3/08; A23L 2/60; A23L 27/30; A23L 33/20

(52) U.S. Cl. CPC C07H 3/08; A23L 2/60; A23L 27/33; A23L 33/20

(57) ABSTRACT

In one aspect, an artificial sweetener compound is disclosed. In another aspect, a sweetened composition includes the sweetener compound. In some aspects, the sweetened composition is combined with a product such as a food product, beverage product, or a pharmaceutical product.

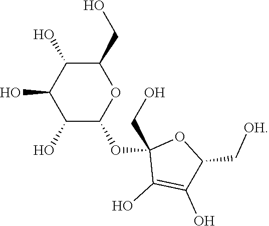 [SMILES: OC[C@H]1O[C@@](CO)(O[C@H]2O[C@H](CO)[C@@H](O)[C@H](O)[C@H]2O)C(O)=C1O]

### CROSS REFERENCE TO RELATED APPLICATION

This application claims priority under 35 U.S.C. § 119(e) to U.S. Application No. 63/540,778, filed Sep. 27, 2023, the disclosure of which is hereby incorporated by reference in its entirety.

### BACKGROUND

Many food and beverage products contain nutritive sweeteners such as sucrose (generally referred to as “sugar” or “table sugar”), glucose, fructose, corn syrup, high fructose corn syrup and the like. Such sweeteners supply not only sweetness to the food and beverage products, but also bulk, texture and desirable functional properties such as browning, humectancy, freezing point depression and the like. They also may produce as favorable sensory response, for example in terms of quality of sweetness, lack of bitterness and off taste, desirable temporal profile and desirable mouthfeel.

Although desirable in terms of taste and functional properties, excess intake of nutritive sweeteners, such as sucrose, has long been associated with an increase in diet-related health issues, such as obesity, heart disease, metabolic disorders and dental problems. This worrying trend has caused consumers to become increasingly aware of the importance of adopting a healthier lifestyle and reducing the level of nutritive sweeteners in their diet.

Excessive intake of dietary sugars can also cause metabolic disorders and induce the increase of inflammatory mediators and certain pro-inflammatory cytokines in various tissues, which leads to insulin resistance and low-grade chronic inflammation. It would be desirable to develop a sweetener that provides favorable functional properties while avoiding the inflammatory effects associated with conventional dietary sugars.

### SUMMARY

According to one aspect, a sweetener compound has the following structure:

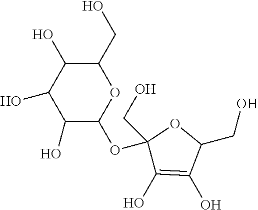 [SMILES: OCC1OC(CO)(OC2OC(CO)C(O)C(O)C2O)C(O)=C1O]

According to one aspect, a sweetener compound has the following structure:

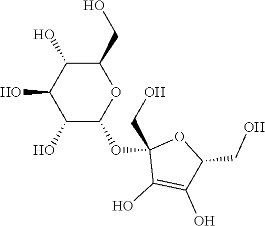 [SMILES: OC[C@H]1O[C@@](CO)(O[C@H]2O[C@H](CO)[C@@H](O)[C@H](O)[C@H]2O)C(O)=C1O]

In another aspect, a sweetened composition comprises the sweetener compound and one or more of a sweet taste improving additive, a bulking agent, a flavoring agent, a stabilizer, or a combination thereof.

In another aspect, a food product comprises the compound disclosed herein or the sweetener composition. In some examples, the sweetener composition is provided as a coating or frosting on a surface of the food product.

In yet another aspect, a beverage product comprises the compound disclosed herein, or the sweetener composition. Other aspects and advantages also are disclosed herein.

In some embodiments, the present disclosure relates to a table-top sweetener comprising the compounds disclosed herein or the sweetener composition.

In some embodiments, the present disclosure relates to a bulking agent comprising the compounds disclosed herein or the sweetener composition.

In some embodiments, the present disclosure relates to a coating agent comprising the compounds disclosed herein or the sweetener composition.

In some embodiments, the present disclosure relates to a cosmetic agent comprising the compounds disclosed herein.

In some embodiments, the present disclosure relates to a pharmaceutical product comprising the compounds disclosed herein or the sweetener composition.

In some embodiments, the present disclosure relates to a nutritional product comprising the compounds disclosed herein or the sweetener composition.

In some embodiments, the present disclosure relates to a sports product comprising the compounds disclosed herein or the sweetener composition.

In some embodiments, the present disclosure relates to a method of making a reduced calorie product comprising adding the compound disclosed herein or the sweetener composition to a food product, a beverage product, a pharmaceutical product, a nutritional product, a sports product, or a cosmetic product.

In some embodiments, the present disclosure relates to a method of making a reduced calorie product comprising adding the compound disclosed herein or the sweetener composition to a food product, a beverage product, a pharmaceutical product, a nutritional product, a sports product, or a cosmetic product.

In some embodiments, the present disclosure relates to a method of increasing bulk of a product comprising adding a bulking agent comprising the compound disclosed herein, or the sweetener composition to the product.

In some embodiments, the present disclosure relates to a method of coating a product comprising applying a coating agent comprising the compound disclosed herein, or the sweetener composition to the product.

### DETAILED DESCRIPTION

According to one aspect, a sweetener compound has the following structure:

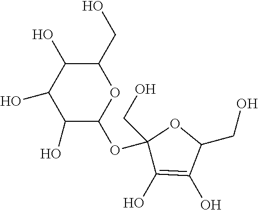 [SMILES: OCC1OC(CO)(OC2OC(CO)C(O)C(O)C2O)C(O)=C1O]

According to one aspect, a compound has the following structure:

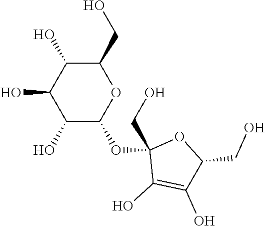 [SMILES: OC[C@H]1O[C@@](CO)(O[C@H]2O[C@H](CO)[C@@H](O)[C@H](O)[C@H]2O)C(O)=C1O]

The compounds described herein which exist in more than one optical isomer form (enantiomer) may be provided either as racemic mixture or by isolating one of the enantiomers, the latter case in which purity as described above may refer to enantiomeric purity.

The compounds may be prepared synthetically using techniques described, for example, in R. Lemieux et al., “A CHEMICAL SYNTHESIS OF SUCROSE,” Journal of the American Chemical Society, 75(16), 4118-4118, with appropriate modifications to reagents to yield the desired structure as will be apparent to persons skilled in the art.

Natural sugars such as sucrose, fructose, and glucose are utilized to provide a pleasing taste to beverages, foods, pharmaceuticals, and oral hygiene/beauty products. Sucrose imparts a taste that is particularly preferred by consumers. Although sucrose imparts excellent sweetening properties, it is caloric. To date, non-caloric or low-calorie sweeteners have been introduced to meet consumer demand. However, sweeteners falling into this class differ from natural caloric sugars in that they struggle with consumer acceptance. On a taste basis, non-caloric or low-calorie sweeteners differ from sugar in terms of temporal profile, maximal response, flavor profile, mouth feel, and/or habituation. In particular, non-caloric or low-calorie sweeteners have a slow onset of sweetness, a long sweet aftertaste, a bitter, metallic, astringent, refreshing and/or licorice-like taste. Non-caloric or low-caloric sweeteners may be synthetic compounds, natural substances, physically or chemically modified natural substances, and/or reaction products derived from synthetic and/or natural substances. There is still a strong demand for natural non-caloric or low-calorie sweeteners with desirable taste characteristics.

In some examples, a sweetener composition comprises the sweetener compound and one or more of a sweet taste improving additive, a bulking agent, a flavoring agent, a stabilizer, or a combination thereof. In some embodiments, the sweetener composition comprises the sweetener compounds disclosed herein and a sweet taste improving additive, a bulking agent, a flavoring agent, and a stabilizer. In some embodiments, the sweetener composition comprises the sweetener compounds disclosed herein and a sweet taste improving additive, and a bulking agent. In some embodiments, the sweetener composition comprises the sweetener compounds disclosed herein and a sweet taste improving additive, a bulking agent, and a flavoring agent. In some embodiments, the sweetener composition comprises the sweetener compounds disclosed herein and a bulking agent, a flavoring agent, and a stabilizer. In some embodiments, the sweetener composition comprises the sweetener compounds disclosed herein and a bulking agent, and a stabilizer.

In one aspect, the sweetener composition may be combined with a cosmetic product, a pharmaceutical product, a nutritional product, or a sports product.

In another aspect, a food product comprises the compound disclosed herein or the sweetener composition. In some examples, the sweetener composition is provided as a coating or frosting on a surface of the food product. In yet another aspect, a beverage product comprises the compound disclosed herein, or the sweetener composition. In some embodiments, the present disclosure relates to a table-top sweetener comprising the compounds disclosed herein or the sweetener composition. In some embodiments, the present disclosure relates to a bulking agent comprising the compounds disclosed herein or the sweetener composition. In some embodiments, the present disclosure relates to a coating agent comprising the compounds disclosed herein or the sweetener composition. In some embodiments, the present disclosure relates to a cosmetic agent comprising the compounds disclosed herein. In some embodiments, the present disclosure relates to a pharmaceutical product comprising the compounds disclosed herein or the sweetener composition. In some embodiments, the present disclosure relates to a nutritional product comprising the compounds disclosed herein or the sweetener composition. In some embodiments, the present disclosure relates to a sports product comprising the compounds disclosed herein or the sweetener composition.

In some embodiments, the present disclosure relates to a method of making a reduced calorie product comprising adding the compound disclosed herein or the sweetener composition to a food product, a beverage product, a pharmaceutical product, a nutritional product, a sports product, or a cosmetic product.

In some embodiments, the present disclosure relates to a method of increasing bulk of a product comprising adding a bulking agent comprising the compound disclosed herein, or the sweetener composition to the product.

In some embodiments, the present disclosure relates to a method of coating a product comprising applying a coating agent comprising the compound disclosed herein, or the sweetener composition to the product.

Non-limiting examples of a food product include a confectionary product, a dessert product such as yogurt, ice cream, biscuits, cakes, a cereal product, baked goods, frozen dairy products, meats, dairy products, condiments, snack bars, soups, dressings, mixes, prepared foods, baby foods, diet preparations, syrups, food coatings, dried fruit, sauces, gravies, jams/jellies, and the like, especially those which are reduced sugar or low sugar products. In some examples, a food product may be an animal feed product. A food product may comprise the sweetener composition as a coating or frosting formed on the surface of the product. The coating may improve the flavor of the food product as well as other properties such as shelf life.

In another aspect, a beverage product comprises the sweetener composition. Non-limiting examples of a beverage product include a carbonated beverage, a non-carbonated beverage, fruit-flavored beverage, fruit-juice, tea, milk, and coffee, especially those which are reduced sugar or low sugar products.

In a further aspect, a table-top sweetener comprising a sweetener composition as described herein.

In yet another aspect, a pharmaceutical product comprises a sweetener composition as described herein. In still another aspect, a nutritional or sports product comprises a sweetener composition as described herein. Another aspect provides a nutritional or sports product comprising a sweetener composition as described herein.

In some examples, a sweetener composition comprises glucose and may further comprise a fruit extract.

Sweetener composition additives may include, for example, saponins, antioxidants, dietary fiber sources, fatty acids, vitamins, glucosamine, minerals, preservatives, hydrating agents, carbon dioxide, probiotics, prebiotics, body weight. One or more functional ingredients such as control agents, osteoporosis control agents, phytoestrogens, long chain primary aliphatic saturated alcohols, phytosterols, and combinations thereof may also be included.

Sweetener compositions include, for example, carbohydrates, polyols, amino acids and their corresponding salts, polyamino acids and their corresponding salts, sugar acids and their corresponding salts, nucleotides, organic acids, inorganic acids, organic salts, including organic acid salts and organic base salts, inorganic salts, bitter compounds, flavoring materials and fragrances, astringent compounds, proteins or protein hydrolysates, surfactants, emulsifiers, flavonoids, alcohols, and polymers.

Consumer health orientation in food is driving increased use of non-caloric high-intensity sweeteners. Although caloric natural sweetener compositions such as sucrose, fructose and glucose provide the most desirable taste to consumers, they are caloric. Many natural and synthetic high-potency sweeteners are non-caloric, but exhibit sweetness with a different temporal profile, maximal perception, flavor profile, body and/or perceptual flexibility than sugar.

It will be appreciated by persons skilled in the art that the amount of a sweetener composition present in a food product, a beverage product, a pharmaceutical product, a nutritional product, a sports product, or a cosmetic product may depend upon the type and amount of sweetener present in the sweetener composition and the desired sweetness of the food or beverage product.

In another aspect, a sweetener composition may be incorporated in a food product, a beverage product, a pharmaceutical product, a nutritional product, a sports product, or a cosmetic product or as a bulking agent or coating agent. The sweetener composition may be formulated in any ingestible form, for example, as a syrup, in powered form, tablet form, tablet form, as granules, in a solution or in any other suitable form including beverages and food products.

Other examples of food products include functional foods, which may provide health benefits such as reducing inflammation.

Functional ingredients may include, by way of non-limiting example, carotenoids, dietary fiber, fatty acids, flavonoids, isothiocyanates, phenols, plant sterols and stanols (phytosterols and phytostanols), polyols, and prebiotics/probiotics. They are classified into categories such as thiols; amino acids; proteins; vitamins; and minerals. Functional ingredients also may be classified based on health benefits such as cardiovascular, cholesterol-lowering, and anti-inflammatory.

Generally, the present sweetener compound may provide functional sweetener compositions having improved temporal and/or flavor profiles.

The compound may be synthesized by chemical modification of sucrose to increase sweetening power. Sucrose is a double sugar of glucose bonded to fructose. It can be prepared through a multi-step manufacturing process to substitute a single bond for a double bond on the sugar molecule fructose. The glucose molecule supplies energy for the brain and muscle cells, while the fructose cell will be altered so that the it will not metabolize and be converted to fat.

While not wanting to be bound by theory, the sweetener compound may bind to T₁r₃ receptors, leading to a sweet sensation within the brain. In some examples, the compound may bind stronger than sucrose, causing a stronger sweetening effect.

While the invention has been described with respect to specific examples, those skilled in the art will appreciate that there are numerous variations and permutations of the above described systems and techniques that fall within the spirit and scope of the invention as set forth in the appended claims.

What is claimed is:

1. A compound having a structure selected from the group consisting of:

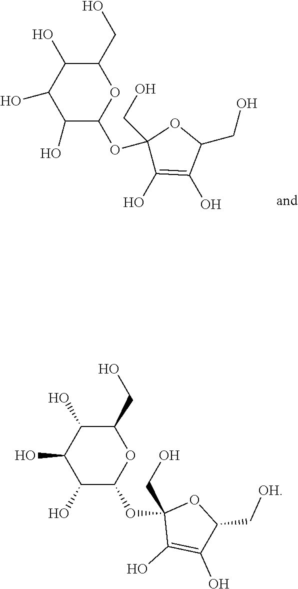 [SMILES: OCC1OC(CO)(OC2OC(CO)C(O)C(O)C2O)C(O)=C1O ; OC[C@H]1O[C@@](CO)(O[C@H]2O[C@H](CO)[C@@H](O)[C@H](O)[C@H]2O)C(O)=C1O]

2. A sweetener composition comprising a compound of claim 1, and one or more of a sweet taste improving additive, a bulking agent, a flavoring agent, a stabilizer, or a combination thereof.

3. A food product comprising a compound of claim 1.

4. The food product of claim 3, wherein the sweetener composition is provided as a coating or frosting on a surface of the food product.

5. A beverage product comprising a compound of claim 1.

6. A table-top sweetener comprising a compound of claim 1.

7. A bulking agent comprising a compound of claim 1.

8. A coating agent comprising a compound of claim 1.

9. A cosmetic agent comprising a compound of claim 1.

10. A pharmaceutical product comprising a compound of claim 1.

11. A nutritional product comprising a compound of claim 1.

12. A sports product comprising a compound of claim 1.

13. A method of making a reduced calorie product comprising adding a compound of claim 1 to a food product, a beverage product, a pharmaceutical product, a nutritional product, a sports product, or a cosmetic product.

14. A method of increasing bulk of a product comprising adding a bulking agent comprising a compound of claim 1 to the product.

15. A method of coating a product comprising applying a coating agent comprising a compound of claim 1 to the product.

16. A compound of claim 1 which has the structure:
